(19) United States
(12) Patent Application Publication
(10) Pub. No.: US 2015/0342728 A1
(43) Pub. Date: Dec. 3, 2015

(54) Accomodating Intraocular Lens with Optical Correction Surfaces

(71) Applicants: Aleksey Nikolaevich SIMONOV, The Hague (NL); Michiel Christiaan ROMBACH, Breda (NL)

(72) Inventors: Aleksey Nikolaevich SIMONOV, The Hague (NL); Michiel Christiaan ROMBACH, Breda (NL)

(21) Appl. No.: 14/350,860

(22) PCT Filed: Oct. 10, 2012
(86) PCT No.: PCT/NL2012/050707

§ 371 Date: Apr. 10, 2014

(30) Foreign Application Priority Data
Oct. 11, 2011 (NL) 2007574
Nov. 9, 2011 (NL) 2007757

(51) Int. Cl. A61F 2/16

(52) U.S. Cl. CPC A61F 2/1624; A61F 2002/1681

(57) ABSTRACT

The present invention provides an accommodating intraocular lens including at least two optical elements and haptics to position the lens in the eye and to transfer movement of driving means. The optical surfaces include cubic free-form surfaces for a variable lens, and additional free-form surfaces to provide a variable correction of at least one variable aberration generated by other optical surfaces of the intraocular lens. Also, optical measures, a prism, and correction of such optical measures, a second prism, included in the design to reduce free-form jump, meaning the elevation of the rim of the cubic free-form surface relative to the base plate, are disclosed.

The present invention relates to an accommodating intraocular lens including at least two optical elements and haptics to position the lens in the eye and to transfer movement of driving means.

Accommodating intraocular lenses with shifting adapted cubic optical surfaces, were disclosed in, for example, WO2005084587, WO2011053143 and US2005324673, which surfaces are largely based on Alvarez surfaces as disclosed, by Louis Alvarez, in U.S. Pat. No. 3,350,294. The optical principles were extended to include rotating designs, semi-rotating designs, designs in which only one optical element has to be shifted and designs with additional fifth-order surfaces for correction of spherical aberrations. These variations are described in U.S. Pat. No. 3,583,790, U.S. Pat. No. 3,350,294 and U.S. Pat. No. 4,650,292 which documents are considered incorporated herein by reference. Such lenses with variable optical power comprise with at least one cubic free-form surface fitted to each optical element, with the combination adapted to provide a variable focus lens of which the degree of focus power depends on the degree of shift, in a direction perpendicular to the optical axis, of at least one cubic free-form surface relative to the other cubic free-form surface. The elementary shape of a cubic free-form surface is best represented by the basic, Alvarez, formula

$$z = \frac{A}{2} ( \frac{{x}^{3}}{3} + {xy}^{2} )$$

which formula also forms the base of two-element accommodating lenses employing lateral movement, movements perpendicular to the optical axis, of the elements.

The accommodating lenses comprise multiple optical elements which are fitted with multiple optical surfaces, which can generate variable and fixed optical distortions which, in turn, can be corrected for by additional optical surfaces for variable and fixed correction. For example, such intraocular lenses with shifting optics can produce various undesired variable aberrations depending on, for example, the distribution of optical surfaces, their mutual degree of movement and other aspects of the optical design. For example, a lens of fixed diopter power can be distributed over two shifting optical elements of an accommodating intraocular lens resulting in a variable astigmatism and coma once the elements shift. Such undesired aberrations of the lens itself can be variably corrected according to the inventions set out below in addition to correction of various undesired variable aberrations of the eye itself. Also, such intraocular lenses with shifting optics can produce various undesired fixed aberrations depending on, for example, tip-tilt of one optical surface which is added to the design to reduce the height of, for example, a cubic free-form surface with regard to the base of the optical element. The present invention discloses additional optical surfaces providing measures to correct for such aberrations generated by optical surfaces of the accommodating intraocular lens.

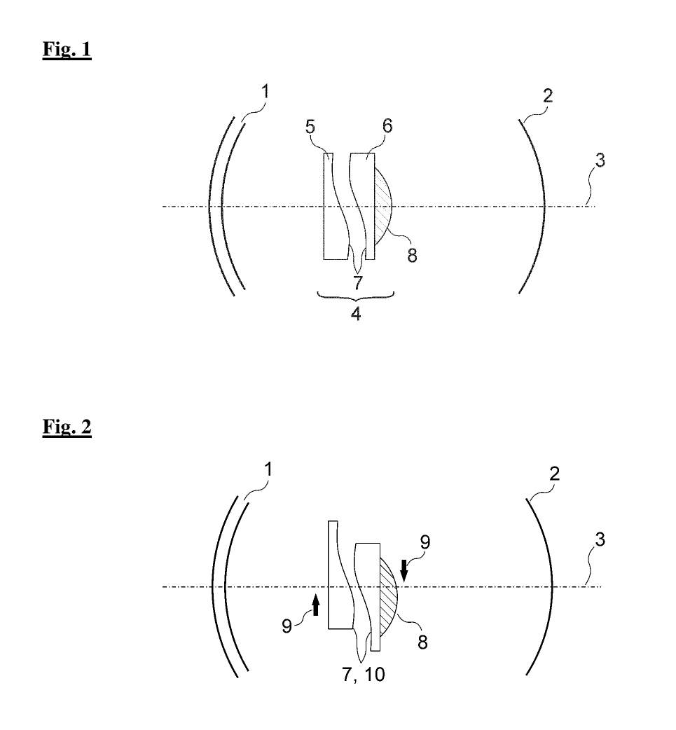

FIG. 1. The eye with the cornea, 1, and the retina, 2, and the optical axis, 3, and the accommodating intraocular lens, 4, with an anterior optical element, 5, and a posterior optical element, 6, comprising free-form optical surfaces including a cubic free-form surface each, 7, and a fixed focus lens, 8. Note that the free-form surfaces are aligned in lateral direction meaning that the focusing power of the variable focus lens is zero.

FIG. 2. For references also refer to FIG. 1. The optical elements are shifted by a distance, 9, which means that the fixed focus lens, 8, is shifted relative to the cornea, 1, perpendicular to the optical axis, 3, by the same distance, which means that the free-form surfaces, 7, should include additional optical surfaces, 10, providing variable correction of at least one variable aberration generated by the shift of said fixed focus lens. Note that the free-form surfaces are shifted in lateral direction meaning that the variable focus lens has a degree of focal power.

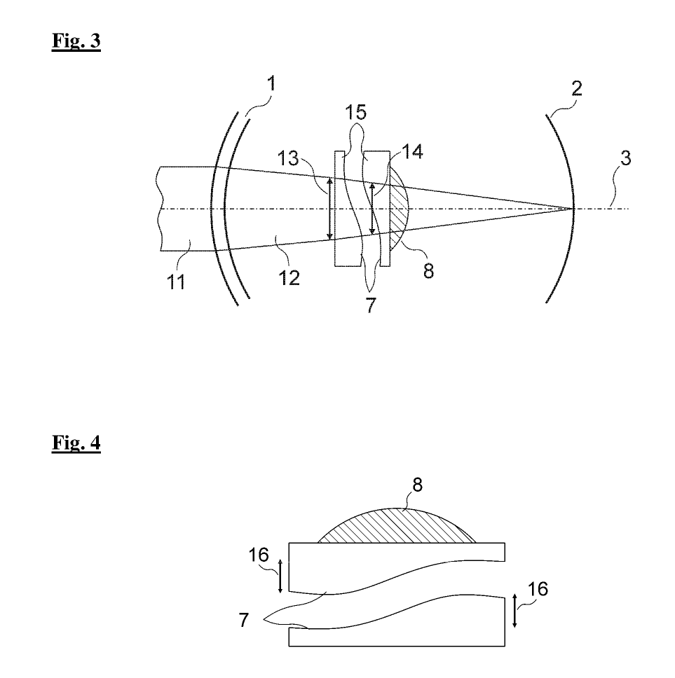

FIG. 3. For references also refer to FIGS. 1 and 2. The incoming light beam, 11, is converted by the cornea, 1, into a converging light beam, 12, which is further focused by the accommodating lens, 4, on the retina, 2. The anterior optical element, 5, receives the beam over a larger area, 13, compared to the area, 14, on the posterior element, 6, because of the convergence of the beam and because of the fact that the cubic free-form surfaces are positioned in separate planes along the optical axis, so the free-form surfaces, 7, should include additional optical surfaces, 15, providing variable correction of at least one variable aberration, for example, correction of variable tilt.

FIG. 4. For references also refer to FIGS. 1-3. The combination of free-form surfaces, 7, produces considerable sags, 16, i.e. peak-to-valley amplitudes.

FIG. 5. For references also refer to FIGS. 1-4. The sags, 16, may be significantly reduced to smaller sags, 17, by tilting one cubic free-form surface in combination with a complimentary tilt of the other cubic free-form surface. Note that the optical elements may also be moved closer, 18, together which benefits overall optical performance of the variable focus lens.

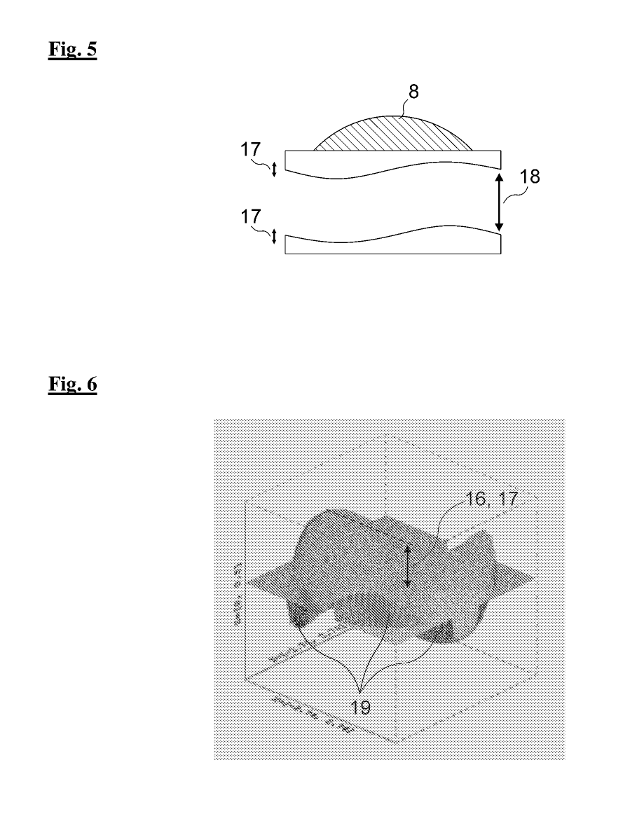

FIG. 6. The figure illustrates the shape of a free-form optical surface with a circular aperture in three dimensions (axonometric drawing). Note the sag, 16, 17, and the free-form jump, 19, meaning the distance from the base plate of an optical element to the circumference of the free-form surface.

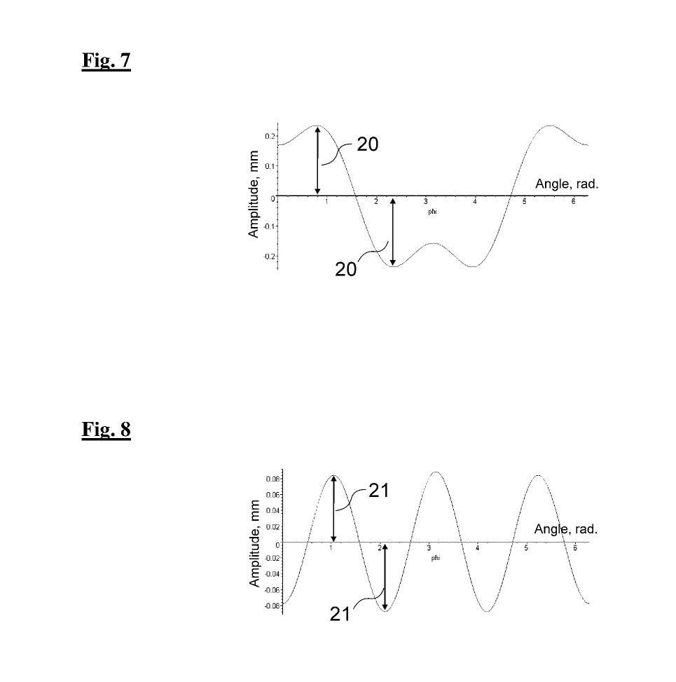

FIG. 7, 8. Tilt of a free-form surface may reduce sag, FIGS. 4, 5, as well as free-form jump. FIG. 7 shows free-form jump of approximately ±0.2 mm, 20, which jump can be largely corrected by tilting of free-form surfaces resulting, in this example, in free-form jump of approximately ±0.08 mm, 21. Reduction of free-form jumps greatly simplifies manufacturing of such free-form surfaces and reduces the overall volume of the accommodating lens which greatly simplifies injection of the lens in the eye. Note that plots in FIGS. 7 and 8 are variations of the circumferential thickness measured in mm versus polar angle measured in radians.

The present invention provides an accommodating intraocular lens comprising at least two optical elements with at least one optical surface fitted to each optical element. The accommodating lens also comprises at least one haptic to position the intraocular lens in the eye and to transfer movement of driving means to at least one of the optical elements. The optical surfaces comprise a combination of two cubic free-form surfaces, with at least one cubic free-form surface fitted to each optical element, with the combination providing a variable focus lens of which the degree of focal power depends on the degree of shift, with shift meaning movement, lateral movement, of at least one cubic free-form surface in a direction perpendicular to the optical axis, of at least one cubic free-form surface relative to at least one other cubic free-form surface. The accommodating lens also comprises at least one fixed focus lens, fitted to at least one of the optical elements to provide refractive correction of the aphakic, lens-less, eye.

The optical elements are fitted with additional optical free-form surfaces comprising a combination of at least two free-form optical surfaces, with at least one such additional free-form surface fitted to each optical element, with such combination providing variable correction of at least one variable aberration generated by a combination of the cornea of the eye and at least one optical surface of the intraocular lens with the degree of correction depending on the degree of shift of at least one such said additional free-form surface on one optical element relative to an other such said additional free-form surface on another element.

In one embodiment of the present invention the optical surfaces include, firstly, for accommodation of the eye, a combination of at least two cubic free-form surfaces, with at least one cubic free-form surface fitted to each optical element, with the combination providing a variable focus lens of which the degree of focus power depends on the degree of shift of at least one cubic free-form surface relative to at least one other cubic free-form surface. The optical surfaces further include, secondly, for correction of fixed refraction of the eye, at least one fixed focus lens, for example, a spherical or parabolic lens or a derivation of such designs, fitted to at least one of said optical elements, or, alternatively, the optical surfaces may include, for example, a combination of a spherical lens and, for example, a toric lens to correct for refraction and for astigmatism.

In another embodiment of the present invention the accommodating intraocular lens further comprises additional optical surfaces fitted to each optical element which additional surfaces including, firstly, a combination of at least two free-form asymmetrical surfaces, meaning surfaces with asymmetry with regard to rotation around the optical axis, with the combination adapted to provide a variable correcting optic of which the degree of correction depends on the degree of shift, in a direction perpendicular to the optical axis, of at least one such free-form surface relative to at least one other such free-form surface, with the variable correcting optic adapted to provide correction of at least one variable aberration generated by at least one other optical surface of the intraocular lens, or, alternatively, by a combination of at least one other optical surface of the intraocular lens and an optical component of the eye, or, alternatively, by at least one component of the eye.

In a further embodiment, at least one additional optical surface may be fitted to each optical element which additional surface includes at least one fixed correction optic, fitted to at least one optical element, with the fixed correction optic adapted to provide correction of at least one fixed aberration generated by at least one other optical surface of the intraocular lens.

The accommodating intraocular lens may comprise a combination of at least two free-form optical surfaces to specifically provide variable correction of at least one variable aberration generated by the shift of said fixed focus lens, a shift relative to the cornea, which shift results in optical misalignment of the optical system of the eye (as illustrated in FIGS. 1 and 2). So, such combination may be optimized to correct for, for example, complex variable aberrations due to shift, perpendicular to the optical axis, of said optical surfaces which correct the fixed refraction of the eye and toric surfaces which correct astigmatisms. Such variable correction surfaces and methods for designing such correction surfaces are largely provided by the prior art document WO2008071760 which document is included, in whole, in the present document by reference.

The surfaces of the cubic free-form surfaces are specified by

$$z = {S}_{A} ( x , y ) = \frac{A}{2} ( \frac{{x}^{3}}{3} + {xy}^{2} ) , \qquad ( 1 )$$

in which A is the constant defining the degree of accommodation of a two-element variable focus lens; x and y are the Cartesian coordinates such that the plane XY coincides with the planes of optical surfaces, the Z axis is chosen along the optical axis.

A fixed, for example, spherical or parabolic, lens may be added to this accommodating surface to correct for the basic refraction of the human eye. For example, such lens may be designed in practice providing, for example, a fixed +22 D refractive power to which an accommodative power of 0-4 D may be added for accommodation. The basic refraction of the eye can be corrected for with a parabolic lens of a fixed optical power with a surface sag given by

z=S_{C}(x,y)=C(x²+y²). (2)

The radius of curvature is (2C)⁻¹ and the focal power of the additional parabolic lens becomes 2C(n−1), where n is the refractive index of the material (W. J. Smith, Modern Optical Engineering, 3rd ed. New York: McGraw-Hill, 2000). In many cases, the corneal aberrations of the eye can be corrected by choosing refractive surfaces of the accommodative IOL in the form:

$$z = S ( x , y ) = {S}_{A} ( x , y ) + \frac{{r}^{2}}{R \{ 1 + \sqrt{1 - ( 1 + k ) \times {( r / R )}^{2}} \}} + {a}_{1} {r}^{4} + {a}_{2} {r}^{6} + … + {a}_{n} {r}^{( 2 n + 2 )} , \qquad ( 3 )$$

where, S_{A} (x, y) provides accommodation and the other terms provide correction of defocus and all higher-order even aberrations including defocus, spherical aberration etc. In Eq. (3) r=\sqrt{x²+y²} is the current radius in polar coordinates; R is the radius of curvature; k is the conic parameter that specifies the type of conicoid; aₙ is the (2n+2)-th order polynomial coefficient which is, in most cases, n≦2. In this formula the simultaneous use of the conic constant and polynomial series is somewhat redundant but has no effect on the operation of the lens. Such an additional surface provides a correction of fixed value and this correction is independent on the variable defocus of the lens. This approach expands the principles described in U.S. Pat. No. 6,609,793 and U.S. Pat. No. 6,705,729 for fixed correction of aberrations in, standard and of fixed focus, intraocular lenses which both describe several aspects of such fixed corrections. Note that Eq. (3) may be extended to the case of odd-order coefficients or coefficients depending only on x and/or y coordinates. In this case correction of any static aberration, for example, astigmatism, can be achieved.

In this document we also describe a varifocal, or variable focus lens with additional variable Zernike terms of which the degree of correction changes along with defocus. In the case of complementary configuration variable third- and higher-order aberrations, expressed in terms of Zernike polynomials as well as their linear combinations are generated having amplitudes changing linearly with the lateral shift Δx. The following base sag function S(x, y) should be used:

$$z = {S}_{Z} ( x , y ) = \frac{A}{2} ( \frac{{x}^{3}}{3} + {xy}^{2} ) + \frac{1}{2} \int {\sum }_{q} {C}_{q} {Z}_{q} ( x , y ) d x \qquad ( 4 )$$

where A is the constant that determines an accommodation amplitude, C_{q} is the modal coefficient corresponding to the q-th Zernike aberration term. Assuming that the elements are made of a material with a refractive index n, the optical path L in the two-element complementary geometry described above, is given by:

L=nh₁+nS_{Z}(x−Δx,y)+h₀+nh₂−nS_{Z}(x+Δx,y), (5)

In this formula the constants h₁, h₂ determine the central thickness of each optical element, and h₀ is the central distance between the respective elements. After simplification, Eq. (5) yields:

$$L = ( {nh}_{1} + {h}_{0} + {nh}_{2} ) - An ( {y}^{2} + {z}^{2} ) Δ x - n Δ x {\sum }_{q} {C}_{q} {Z}_{q} ( x , y ) + nR ( x , y , Δ x ) \qquad ( 6 )$$

and the corresponding optical path difference (OPD) takes the form:

$$OPD = ( n - 1 ) ( {h}_{1} + {h}_{2} ) - A ( n - 1 ) ( {y}^{2} + {z}^{2} ) Δ x - ( n - 1 ) Δ x {\sum }_{q} {C}_{q} {Z}_{q} ( x , y ) + ( n - 1 ) R ( x , y , Δ x ) . \qquad ( 7 )$$

As seen from Eq. (7), the OPD produced by a two-element variable focus lens with optical surfaces according to Eq. (4) moving laterally by Δx each contains:
constant term, (n−1)(h₁+h₂), which is a constant piston mode Z₀;
variable focus term, A(n−1)(y²+z²)Δx, which is a variable defocus mode Z₄; the optical power of the corresponding variable lens is F⁻¹=2A(n−1)Δx;

$$( n - 1 ) Δ x {\sum }_{q} {C}_{q} {Z}_{q} ( x , y ) ,$$

variable Zernike terms, which is a linear combination of Zernike aberration modes Z_{q} with variable aberration amplitudes (n−1)ΔxC_{q}; additional optical power (in diopters) produced by the defocus term C₄ is F⁻¹=2\sqrt{3}C₄(n−1)Δx;
variable residual term, (n−1)R(x, y, Δx), which is a contribution of higher-order (non-linear) shift-dependent contributions Δx³, Δx⁵ ect. When |Δx|<<1, these contributions are negligibly small and can be omitted for practical purposes.

So, a pair of refractive elements, shaped according the base function S_{Z} (x, y) given above, provides linear change of the specified optical aberrations along with defocus/accommodation for use in accommodating lenses for treatment of cataracts, presbyopia and spectacle-replacement in general. As mentioned above, correction of constant ocular refractive errors can be accomplished by adding to one of the optical element of the IOL fixed parabolic, spherical and aspherical surfaces according to Eqs. (2-3).

When applied to accommodating lenses we assume that ocular aberrations expressed in terms of Zernike polynomials are accommodation-dependent, in our case shift-dependent, and associated with the change of the shape of the cornea and lateral shift of the optical elements. In the later case, for example, when a fixed focus lens moves transversely with respect to the cornea of the eye, it produces shift-dependent astigmatism, coma etc. Such ocular aberrations can be corrected simultaneously with defocus by using e.g. a two-element accommodative intraocular lens with the specified above additional refractive surfaces shaped according to Eq. (4).

Such two-element accommodating lenses produce variable defocus aberration which is linearly changing with shift Δx. Reciprocal shift of the two refractive elements carrying surfaces S_{Z} (x, y) by Δx in the opposite direction perpendicular to the optical axis results in a linear change of the focusing power

F⁻¹=2A(n−1)Δx+2\sqrt{3}C₄(n−1)Δx, (8)

where A is the cubic term amplitude and C₄ is the magnitude of additional defocus.

As follows from Eq. (7), modal amplitudes of aberration terms change linearly with shift Δx. Reciprocal shift of the two refractive elements with the surfaces according to Eq. (7) by Δx in the opposite directions perpendicular to the optical axis results in a linear change of the q-th Zernike aberration term (excluding defocus, i.e. q≠4). The new modal amplitudes C′_{q} become:

C′_{q}=(n−1)ΔxC_{q}. (9)

Correction of defocus results in a simultaneous variation of a linear combination of aberrations. Reciprocal shift of the two refractive elements with the profiles S_{Z} (x, y), specified above, by Δx in the opposite directions perpendicular to the optical axis gives rise to the linear change of the combination of Zernike aberration terms:

$$Z = {\sum }_{q} {C}_{q}^{′} {Z}_{q} ( x , y ) , \qquad ( 10 )$$

Where, according to Eq. (9), C_{q}′=(n−1)ΔxC_{q} are the new modal amplitudes. The relative weights of monochromatic aberrations, i.e. Zernike aberration modes, can be adjusted as required by choosing the corresponding constant coefficients C_{q}.

All embodiments described in this document may also have GRIN and also Fresnel designs in addition to a traditional lens design. GRIN and Fresnel designs allow lenses to be manufactured significantly thinner compared to traditional lenses and the degree of chromatic aberrations can be reduced by Fresnel designs and GRIN designs offer alternatives with regard to distribution of optical quality over the surface of the optics.

For example, simultaneous correction of variable defocus and astigmatism can be achieved by using two-element variable focus lens with the optical surfaces given by:

$$z = S ( x , y ) = \frac{A}{2} ( \frac{{x}^{3}}{3} + {xy}^{2} ) + B \{ \frac{{x}^{3}}{3} - xy \} , \qquad ( 11 )$$

where A and B are constant coefficients. In turn, simultaneous correction of variable defocus and coma can be obtained with the surfaces:

$$z = S ( x , y ) = \frac{A}{2} ( \frac{{x}^{3}}{3} + {xy}^{2} ) + C \{ \frac{3}{4} {x}^{4} + \frac{3}{2} {x}^{2} {y}^{2} - {x}^{2} \} , \qquad ( 12 )$$

where A and C are constant coefficients. Note that, in practice, correction of the aberrations generated by an optical surface or by combinations of optical surfaces of the accommodating lens, or by combinations of optical surfaces of the accommodating lens and an optical component of the eye, aberrations such as defocus, spherical aberration, astigmatism and coma will provide the eye with a virtually aberration-free vision. However, if needed, formulas for correction of all variable corrections can be derived within the framework outlined above.

In one embodiment of the present invention the accommodating lens may comprise a combination of at least two free-form optical surfaces to provide variable correction of at least one variable aberration generated by the cubic free-form surfaces being positioned in separate planes along the optical axis (for illustration see FIG. 3). The incoming light beam is converted, focused, by the cornea into a converging light beam which is further focused by the accommodating lens on the retina. Because of the convergence of the light beam, and because of the fact that the cubic free-form surfaces are positioned in separate planes along the optical axis, the anterior optical element of the accommodating lens receives the beam over a larger area compared to the area on the posterior element. To correct for undesired aberrations such as variable tilt caused by said disparity in area the free-form surfaces may include additional optical surfaces to provide variable correction of at least one such variable aberration. The degree of aberration depends on design specifications of the accommodating lens, for example, the space between the optical elements.

The accommodating lens according to the present invention may further comprise a combination of free-form surfaces which, in turn, comprise a combination of tilts including complementary tilt of one cubic free-form surface to provide correction of at least one variable aberration generated by tilt of the other cubic free-form surface (see also FIGS. 4-8). Such combination of tilts can minimize surface sags of the cubic free-form surfaces and therefore minimize overall thickness of the accommodating lens. The tilt of one optical surface may be part of the design to reduce the height of, for example, a cubic free-form surface and additional free-form surfaces with regard to the base of the optical element, also termed: free-form jump, which measure, in turn, reduces the thickness of the intraocular lens. So, the intraocular lens may comprise two combinations of a free-form surface, each tilted, with such combination fitted to each optical element, which combinations adapted to minimize the thickness, in the direction of the optical axis, of the cubic free-form surface, and reduction of free-form jump, meaning reduction of the distance from the base plate of an optical element to the starting point of the free-form surface.

Such intraocular lens may provide correction of at least one optical aberration of the eye, for example, fixed refractive error, fixed refraction error of the eye, or, for example, a combination of such refractive error and astigmatism. The lens may comprise at least one additional optical surface adapted to provide correction of astigmatism of the cornea. So, the intraocular lens comprises at least one optical surface providing correction of at least one optical aberration of the eye, which may be correction of at least one fixed optical aberration of the eye, with the fixed optical aberration of the eye a refractive correction of the eye, and, in addition, the intraocular lens provides correction of at least one variable optical aberration of the eye, which main variable aberration is variable focus aberration, other variable aberrations being generated by the lens itself. The lens replaces the natural lens of the eye by surgical implant in the eye. An accommodating intraocular lens as disclosed in the present document is intended to replace the natural lens of the eye, to, for example, treat presbyopia.

In another embodiment of the present invention, the intraocular lens may comprise at least one additional optical surface adapted to provide correction of astigmatism of the cornea. The intraocular lens may comprise at least one additional optical surface adapted to provide correction of a fixed astigmatism, or, alternatively, the at least one additional optical surface is adapted to provide correction of a variable astigmatism.

In another embodiment of the present invention the accommodating intraocular lens is adapted to replace the natural lens of the eye with the lens comprising at least two optical elements with at least one optical surface fitted to each optical element, and with at least one haptic adapted to position the intraocular lens in the eye and to transfer movement of driving means to at least one of the optical elements, with the optical surfaces comprising a combination of two cubic free-form surfaces, with at least one such cubic free-form surface fitted to each optical element, with the combination adapted to provide a variable focus lens of which the degree of focal power depends on the degree of shift, with shift meaning movement of at least one cubic free-form surface in a direction perpendicular to the optical axis, of at least one cubic free-form surface relative to at least one other cubic free-form surface, and at least one fixed focus lens, fitted to at least one of the optical elements, with the fixed focus lens adapted to provide basic refractive correction of the aphakic eye, with the optical elements fitted with a combination of at least two additional free-form optical surfaces, with at least one such additional free-form surface fitted to each optical element, with the combination adapted to provide variable correction of at least one variable aberration generated by a combination of the cornea of the eye and at least one optical surface of the intraocular lens with the degree of correction depending on the degree of shift of at least one such said additional free-form surface on one optical element relative to at least one other such said additional free-form surface on at least one other element, the combination of at least two free-form optical surfaces may be adapted to provide variable correction of at least one variable aberration generated by the shift of said fixed focus lens, in a direction perpendicular to the optical axis on which the cornea remains in a fixed position, for example the combination of at least two additional free-form optical surfaces may be adapted to provide variable correction of at least one variable aberration generated by the cubic free-form surfaces being positioned in separate planes along the optical axis, or, alternatively, the combination of additional free-form surfaces may comprises a combination of tilts comprising complementary tilt of the other cubic free-form surface adapted to provide correction of at least one variable aberration generated by tilt of the other cubic free-form surface, with, for example, the combination of tilts adapted to minimize surface sags of the cubic free-form surfaces, also, the intraocular lens may comprise at least one additional optical surface adapted to provide correction of astigmatism of the cornea, which may be correction of a fixed astigmatism, or, alternatively, correction of a variable astigmatism.

1. An accomodating intraocular lens, adapted to replace the natural lens of the eye, with the lens comprising at least two optical elements with at least one optical surface fitted to each optical element, and with at least one haptic adapted to position the intraocular lens in the eye and to transfer movement of driving means to at least one of the optical elements, with the optical surfaces comprising:
a combination of two cubic free-form surfaces, with at least one such cubic free-form surface fitted to each optical element, with the combination adapted to provide a variable focus lens of which the degree of focal power depends on the degree of shift, with shift meaning movement of at least one cubic free-form surface in a direction perpendicular to the optical axis, of at least one cubic free-form surface relative to at least one other cubic free-form surface,
at least one fixed focus lens, fitted to at least one of the optical elements, with the fixed focus lens adapted to provide basic refractive correction of the aphakic eye,
characterized in that the optical elements are fitted with a combination of at least two additional free-form optical surfaces, with at least one such additional free-form surface fitted to each optical element, with the combination adapted to provide variable correction of at least one variable aberration generated by a combination of the cornea of the eye and at least one optical surface of the intraocular lens with the degree of correction depending on the degree of shift of at least one such said additional free-form surface on one optical element relative to at least one other such said additional free-form surface on at least one other element.

2. The intraocular lens according to claim 1, wherein the combination of at least two free-form optical surfaces is adapted to provide variable correction of at least one variable aberration generated by the shift of said fixed focus lens, in a direction perpendicular to the optical axis on which the cornea remains in a fixed position.

3. The intraocular lens according to claim 1, wherein the combination of at least two additional free-form optical surfaces adapted to provide variable correction of at least one variable aberration generated by the cubic free-form surfaces being positioned in separate planes along the optical axis.

4. The intraocular lens according to claim 1, wherein the combination of additional free-form surfaces comprises a combination of tilts comprising complementary tilt of the other cubic free-form surface adapted to provide correction of at least one variable aberration generated by tilt of the other cubic free-form surface.

5. The intraocular lens according to claim 4, wherein the combination of tilts is adapted to minimize surface sags of the cubic free-form surfaces.

6. The intraocular lens according to claim 1, wherein the lens comprises at least one additional optical surface adapted to provide correction of astigmatism of the cornea.

7. The intraocular lens according to claim 6, wherein the additional optical surface is adapted to provide correction of a fixed astigmatism.

8. The intraocular lens according to claim 6, wherein the additional optical surface is adapted to provide correction of a variable astigmatism.
